(19) United States
(12) Patent Application Publication
(10) Pub. No.: US 2019/0362813 A1
(43) Pub. Date: Nov. 28, 2019

(54) System and Method for Identifying Connections Between Perturbagens and Genes Associated with a Skin Condition

(71) Applicant: The Procter & Gamble Company, Cincinnati, OH (US)

(72) Inventors: Wenzhu NMN ZHAO, Mason, OH (US); Tomohiro NMN HAKOZAKI, Cincinnati, OH (US); Leo Timothy LAUGHLIN, II, Mason, OH (US)

(21) Appl. No.: 16/419,521

(22) Filed: May 22, 2019

Related U.S. Application Data
(60) Provisional application No. 62/674,840, filed on May 22, 2018.

(51) Int. Cl. G16B 40/20; C12Q 1/6837; G01N 33/50; G16H 50/70; G16B 5/00; G16B 20/00; G16B 30/00

(52) U.S. Cl. CPC G16B 40/20; C12Q 1/6837; G01N 33/5023; G01N 33/5044; G01N 2800/207; G16H 50/70; G16B 5/00; G16B 20/00; G16B 30/00; G01N 33/5091

(57) ABSTRACT

An improved connectivity mapping method for identifying connections between a potential skin care agent associated with an instance and genes associated with a skin hyperpigmentation condition. The system includes a non-transitory computer readable medium having a plurality of instances stored thereon, and a biased gene expression signature associated with a skin condition. The biased condition signature is constructed by filtering an unbiased condition signature through a benchmark signature.

### TECHNICAL FIELD

The present disclosure is directed generally to systems and methods of identifying potential skin care agents using a biased connectivity mapping technique. More specifically, the present disclosure is directed to improving the accuracy of connectivity mapping techniques for identifying potential skin care agents by biasing transcriptional gene selection with gene expression signature data from a benchmark material.

### BACKGROUND

Skin conditions include some of the most common cosmetic disorders treated in the developing world, which has led to a multi-billion-dollar cosmetic skin care industry. Commonly treated skin conditions include fine lines and/or wrinkles; hyperpigmentation; uneven skin tone; sallowness; dullness; redness; poor barrier properties (e.g., from the thinning of one or more layers of skin, reduced elasticity and/or reduced resiliency); enlarged pores; oily, shiny, and/or dull appearance; acne; dryness; itchiness; flakiness; and poor exfoliation or desquamation. Different skin conditions are associated with widely varied triggers, biological mechanisms, environmental factors, and clinical manifestations, complicating research into the identification of suitable active agents and treatments. For example, disorders of skin pigment production and distribution can occur as a function of intensity and duration of UV radiation exposure, life style habits, chronological age, endocrine functioning and disease state.

Despite the prevalence of cosmetically treated skin conditions and research efforts to identify their causes, the underlying mechanisms responsible for many skin conditions remain unclear. It is not uncommon for the pathogenesis of a particular condition to be multifactorial, and the complex etiologies associated with a skin condition can be influenced by a combination of genetic and environmental factors unique to each condition. Thus, there is a persistent need in the art for systems and methods that can help identify actives for treating a specific skin condition.

Recently, a technique known as connectivity mapping, or “CMap,” has been found to be a useful high-throughput screening tool for identifying new skin care actives. CMap is an in silico hypothesis generating and testing tool that links gene regulation to an active agent. The general notion that functionality can be accurately determined for previously uncharacterized genes, and that potential targets of drug agents can be identified by mapping connections in a database of gene expression profiles for drug-treated cells, was spearheaded in 2000 with publication of a seminal paper by T. R. Hughes, et al. (“Functional Discovery via a Compendium of Expression Profiles” Cell 102, 109-126 (2000)), followed shortly thereafter with the launch of the Connectivity Map Project by Justin Lamb, et al. (“Connectivity Map: Gene Expression Signatures to Connect Small Molecules, Genes, and Disease,” Science, Vol 313, 2006).

U.S. Pat. No. 9,434,993 and U.S. Publication Nos. 2015/0292018, 2013/0261007, 2013/0259816, 2013/0261006, 2013/0261024A1, and 2017/0343534 disclose examples of systems and methods for using CMap to identify potential actives for treating various conditions associated with unhealthy keratinous tissue. CMap techniques for identifying potential skin care agents generally rely on the use of transcriptional gene expression data generated by selecting representative genes from a gene expression profile based on a statistical model. A gene expression signature can be generated using a messenger RNA (“mRNA”) expression profile obtained from a skin biopsy or other skin tissue sample in which a skin condition is present (e.g., hyperpigmented spot). This is commonly referred to as a “condition signature.” Alternatively, the gene expression signature may be generated using an mRNA expression profile from cells treated with one or more benchmark actives (i.e., an active known to cause a particular effect in the subject cells). This is commonly referred to as a “benchmark signature.”

Using the condition or benchmark gene expression signature, a user can query a library of materials that have been used to treat a representative cell line and the resulting mRNA expression profile measured. The actives and their corresponding profiles are represented as CMap instances in a CMap database. The effectiveness of a test agent to reverse a skin condition or the similarity of a test agent's action to a benchmark is ranked by its CMap scores, which are calculated by checking how the genes in the signature are modulated by the test agent. If the reference signature comes from a condition signature, then materials that have an opposite correlation score to the signature (that is materials that reverse transcriptional changes associated with the skin condition of interest) are desired. If the reference signature comes from a benchmark signature, then materials that have the highest positive correlation to the reference signature are desired, since the user is looking for activity along the same direction as the benchmark.

One drawback to current CMap techniques in the cosmetic filed is that the success rate of using condition signatures can be much worse than using benchmark signatures. One reason for this may be that using a tissue sample with multiple cell types to generate a gene expression signature can introduce “noise” into the system. That is, not every transcriptional change in the condition signature may be directly related to the attributes of the condition that involve therapeutic modulation.

Accordingly, there is a need to improve the accuracy of CMap techniques for identifying potential skin actives by biasing a condition signature with data from a benchmark signature.

### SUMMARY

Disclosed herein is a method of improving the predictability of a connectivity mapping method for identifying potential skin care agents. The method comprises constructing a condition signature and benchmark signature from a skin tissue sample. The condition signature is then filtering through the benchmark signature using a computer algorithm to provide a biased condition signature. The biased condition signature comprises a plurality of up-regulated genes and a plurality of down-regulated genes that all have a p-value of 0.1 or less. The biased condition signature is used to query a database of instances in which each instance is associated with a skin care agent. A connectivity score is generated for each instance, and the skin care agent associated with an instance in the database is identified as a potential skin care agent when the connectivity score of the instance has a negative correlation to the biased condition signature.

### BRIEF DESCRIPTION OF THE FIGURES

FIG. 1 is a schematic illustration of an example of constructing a condition signature from a test sample.

FIG. 2 is a schematic illustration of an example of constructing a benchmark signature from a control sample.

FIG. 3 is a schematic illustration of an example of the present system.

FIG. 4 is a schematic illustration of an example of an instance.

FIGS. 5, 6, and 7 illustrate examples of correlating and rank ordering connectivity scores.

### DETAILED DESCRIPTION

CMap techniques can be useful for identifying potential new skin care agents, but the success rate when using a condition signature is typically not as high as the success rate of using a benchmark signature. It is believed, without being limited by theory, that condition signatures obtained from skin tissue samples may introduce “noise” into a gene expression signature due to the presence of more than one type of cell (e.g., keratinocytes, fibroblasts, melanocytes) in the sample. That is, not every transcriptional change in the condition signature may be directly related to the attributes of the condition that involve therapeutic modulation. In contrast, benchmark signatures are typically obtained from a commercially available cell line (e.g., tert-keratinocytes, BJ fibroblasts, B16 melanoma cells), which may provide a gene expression signature that is more relevant to the therapeutic modulation of a skin condition of interest. Surprisingly, it has now been discovered that biasing a condition signature with benchmark signature data can improve the likelihood that a potential skin care agent identified using a CMap technique will be effective for treating the skin condition of interest. Thus, the present discovery improves the predictability of previous CMap systems.

Unless otherwise defined, all technical and scientific terms used herein have the same meaning as commonly understood by one of ordinary skill in the art to which the disclosure pertains. As used herein, the singular forms “a,” “an,” and “the” are intended to include the plural forms as well, unless the context clearly indicates otherwise. Unless otherwise indicated, the disclosure of any ranges in the specification and claims are to be understood as including the range itself and also anything subsumed therein, as well as endpoints. All numeric ranges are inclusive of narrower ranges; delineated upper and lower range limits are interchangeable to create further ranges not explicitly delineated.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

The systems, methods, and devices herein can comprise, consist essentially of, or consist of, the essential components as well as optional ingredients described herein. When referring to a transcriptional profile, “consisting essentially of” means that the transcriptional profile only includes data related to the transcription of specific genes selected from a subject gene expression signature or gene expression profile, except that it may include additional data that does not materially alter the basic and novel characteristics of the claimed method or system (e.g., metadata). The genes and proteins disclosed herein correspond to their respective known sequences as of May 22, 2018.

“Benchmark agent” means a skin care agent or combination of skin care agents known to induce or cause a known effect (positive or negative) on skin tissue. In various embodiments, the effect of the benchmark agent is a robust, desired effect on a cell type or tissue of interest. Some non-limiting examples of benchmark skin care agents that may be suitable for use herein include niacinamide, resorcinol, kojic acid, arbutin, deoxy-arbutin, vitamin C compounds, vitamin E compounds, sulfhydryl compounds, ellagic acid, glucosamine, N-acetyl glucosamine, tunicamycin, protease inhibitors, N-undecylenoyl phenylalanine, retinoids (trans-retinoic acid, retinol, retinaldehyde, etc.), hexamidine, fluocinolone acetonide, hydroquinone, tretinoin, hydrocortisone, phytosterol, glycyrrhetinic acid, tranexamic acid, chamomile extract, salicylic acid, alpha hydroxy acids, alpha-keto acids, and adenosine monophosphate, a mixture of fluocinolone acetonide, hydroquinone, and tretinoin, and combinations thereof. A particularly suitable example of a benchmark skin care agent is Tri-luma® brand skin cream, available with a prescription from Galderma, which contains a mixture of fluocinolone acetonide (0.01%), hydroquinone (4%), and tretinoin (0.05%).

“Benchmark signature” means a gene expression signature constructed using gene expression profile data obtained from skin tissue or skin cells that have been treated with a benchmark skin care agent.

“Computer readable medium” refers to any non-transitory, electronic storage medium implemented in any method or technology for storage of information such as computer readable instructions, data and data structures, digital files, software programs and applications, or other digital information. Some non-limiting examples of computer readable media include an application-specific integrated circuit (ASIC), a compact disk (CD), a digital versatile disk (DVD), random access memory (RAM), synchronous RAM (SRAM), dynamic RAM (DRAM), synchronous DRAM (SDRAM), double data rate SDRAM (DDR SDRAM), direct RAM bus RAM (DRRAM), read only memory (ROM), programmable read only memory (PROM), electronically erasable programmable read only memory (EEPROM), and a removable flash memory device (e.g., memory stick or thumb drive).

“Connectivity map” and “CMap” are interchangeable and refer broadly to devices, systems, articles of manufacture, and methodologies for identifying relationships between cellular phenotypes or skin conditions, gene expression, and perturbagens.

“Connectivity score” refers to a derived value representing the degree to which an instance correlates to a query.

“Control sample” means a matched sample (e.g., the same cell and/or tissue type used to generate the transcriptional data for a corresponding test sample) that is not afflicted with the subject skin condition and/or has not been treated with a perturbagen. A control gene expression profile can also be derived from prediction algorithms or computed indices from population studies. The control sample may be matched for race, gender, age, geographic location, and/or ethnic origin.

“Data architecture” refers generally to one or more digital data structures comprising an organized collection of data. In some embodiments, the digital data structures can be stored as a digital file (e.g., a spreadsheet file, a text file, a word processing file, a database file, etc.) on a computer readable medium. In some embodiments, the data architecture is provided in the form of a database that may be managed by a database management system (DBMS) that is be used to access, organize, and select data (e.g., instances and gene expression signatures) stored in a database.

“Dermatologically acceptable carrier” means a carrier that is suitable for topical application to the keratinous tissue. The dermatologically acceptable carrier may be in a wide variety of forms such as, for example, simple solutions (water-based or oil-based), solid forms (e.g., gels or sticks) and emulsions.

“Differentially expressed” means there is an observed difference or change in expression levels between two experimental conditions that is statistically significant. For example, a gene that is up-regulated in a test sample relative to a control sample is differentially expressed when the difference in expression level is statistically significant (e.g., p≤0.1 or p≤0.05). In another example, a gene that is down-regulated in a test sample relative to a control sample is differentially expressed when the difference in expression level is statistically significant.

“Effective amount” refers to the amount of a skin composition sufficient to significantly induce a positive benefit to skin, such as a health, appearance, and/or feel benefit relevant to a skin condition of interest, but low enough to avoid serious side effects (i.e., to provide a reasonable benefit to risk ratio, within the scope of sound judgment of the skilled artisan).

“Gene expression profile” or “gene expression profiling experiment” refer to the measurement of the expression of multiple genes in a biological sample using any suitable profiling technology. For example, the mRNA expression of thousands of genes may be determined using microarray techniques. Other emerging technologies that may be used include RNA-Seq or whole transcriptome sequencing using NextGen sequencing techniques.

“Gene expression signature” means a rationally derived list, or plurality of lists, of genes having an expression pattern that is representative of a skin condition or a biological response to a perturbagen. A gene expression signature generally comprises a combination of genes whose expression, relative to a normal or control state, is increased (up-regulated) and/or decreased (down-regulated), that may serve as proxy for a phenotype of interest. Generally, a gene expression signature for a modified cellular phenotype (e.g., a phenotype observed in response to exposure to a perturbagen or biological challenge or phenotype associated with a skin condition) may be described as a set of genes differentially expressed in the modified cellular phenotype over a control (i.e., wild-type or unaffected cellular phenotype). A gene expression signature can be derived from various sources of data, including but not limited to, in vitro testing, in vivo testing, database information, and combinations thereof. In various embodiments, data associated with a gene expression signature comprises an ordered list of “identifiers” representing differentially expressed genes. Exemplary identifiers include, but are not limited to, gene names, gene symbols, microarray probe set ID values, and combinations thereof. Optionally, a gene expression signature comprises a first list of identifiers representative of a plurality of up-regulated genes of the condition(s) of interest and a second list

“Instance” mean data from a gene expression profiling experiment in which skin cells are dosed with a perturbagen. In some embodiments, the data comprises a list of identifiers representing the genes that are part of the gene expression profiling experiment. The identifiers may include gene names, gene symbols; microarray probe set IDs, or any other identifier. In some embodiments, an instance may comprise data from a microarray experiment and comprises a list of probe set IDs of the microarray ordered by their extent of differential expression relative to a control. The data may also comprise metadata, including but not limited to data relating to one or more of the perturbagen, the gene expression profiling test conditions, the skin cells, and the microarray.

“Microarray” refers broadly to any ordered array of nucleic acids, oligonucleotides, proteins, small molecules, large molecules, and/or combinations thereof on a substrate that enables gene expression profiling of a biological sample. Non-limiting examples of microarrays are available from Affymetrix, Inc.; Agilent Technologies, Inc.; Illumina, Inc.; GE Healthcare, Inc.; Applied Biosystems, Inc.; Beckman Coulter, Inc.; etc.

“Perturbagen” means a chemical or physical stimulus that evokes a biological response in skin tissue, leading to a shift in gene expression from normal or wild-type gene expression. Any substance, chemical, compound, small or large molecule, active, natural product (e.g., chemokine), extract, and combination thereof can be employed as a perturbagen. “Perturbagen” also includes any other stimulus that generates differential gene expression data, such as, for example, UV radiation, heat, osmotic stress, pH, a microbe, a virus, a recombinant cytokine or growth factor, or small interfering RNA. A perturbagen may be, but is not required to be, a skin care agent. In some embodiments, the perturbagen is applied to skin cells and gene expression is measured. The resulting transcriptional data can be stored, e.g., as an instance in a data architecture.

“Query” refers to data that is used as an input to a Connectivity Map and against which a plurality of instances are compared. A query may include a gene expression signature associated with a skin condition or an instance. A CMap may be queried with perturbagens, gene expression signatures, skin disorders, thematic signatures, or any data feature or combination of data features or associations that are included in the data architecture.

“Skin care agent” means any substance, as well as any component thereof, which may be safely and effectively rubbed, poured, sprinkled, sprayed, introduced into, or otherwise topically applied to skin to cause a desired change in skin condition (e.g., health, feel, and/or appearance). Some non-limiting examples of skin care agents can be found in: the PubChem database associated with the National Institutes of Health, USA, the Ingredient Database of the Personal Care Products Council, the 2010 International Cosmetic Ingredient Dictionary and Handbook, 13^{th} Edition, published by The Personal Care Products Council, the SkinDeep database. Other non-limiting examples of skin care agents include botanicals (i.e., materials derived from one or more of a root, stem bark, leaf, seed or fruit of a plant). Another category of skin care agents are vitamin compounds and derivatives and combinations thereof, such as a vitamin B3 compound, a vitamin B5 compound, a vitamin B6 compound, a vitamin B₉ compound, a vitamin A compound, a vitamin C compound, and/or a vitamin E compound (e.g., retinol, retinyl esters, niacinamide, folic acid, panthenol, ascorbic acid, tocopherol, and tocopherol acetate). Other non-limiting examples of skin care agents include sugar amines, phytosterols, hexamidine, hydroxy acids, ceramides, amino acids, and polyols.

“Skin condition” means any skin phenotype of interest, including an abnormal phenotype associated with disease, biological disorder, malnutrition, age, and infection.

Disclosed herein are devices, systems and methods for constructing a biased gene expression signature, which can be used to improve the predictability of a CMap query as compared to its unbiased counterpart. The biased gene expression signature can be used for predicting the influence of test agents on a skin condition of interest. Constructing an unbiased gene expression signature according to the present method generally includes (a) measuring gene expression in test sample (e.g., a skin tissue sample, primary cells, or a skin cell line); (b) identifying genes differentially expressed in the test sample by comparing the gene expression measurement of (a) with a gene expression measurement for a control sample; (c) calculating a gene expression consistency value that is representative of the significance of the differential gene expression identified in (b); (d) creating an ordered list of the differentially expressed genes based on their gene expression consistency value; (e) biasing the ordered list of differentially expressed genes with a gene expression measurement from a benchmark agent. It is to be appreciated that one or more steps in the method may be conducted using a programmable computer. Some non-limiting examples of methods, systems, and devices for measuring gene expression, identifying differentially expressed genes, calculating gene expression consistency values, creating an ordered list of gene identifiers, and general connectivity mapping techniques are described in U.S. Pat. No. 9,434,993 and U.S. Publication Nos. 2013/0261007 and 2017/0343534.

In certain embodiments, a computer is used to query a data architecture of stored skin instances with a biased gene expression signature. Each skin instance is associated with a skin care agent. The querying comprises comparing the biased gene expression signature to each stored skin instance. The in silico method facilitates identification of skin care agents that induce a statistically significant change in expression of a statistically significant number of genes associated with a skin condition of interest leading to the identification of new cosmetic agents for treating skin conditions or new uses for known cosmetic agents. While the present disclosure generally refers to skin pigmentation conditions (e.g., hyperpigmentation, age spots, lentigines, melasma), it is to be appreciated that the present methods and systems can be applied to any skin condition of interest.

A method for formulating a skin care composition is also provided herein. The method generally includes accessing a plurality of instances stored on a computer readable medium, accessing a biased gene expression signature stored on a computer readable medium, comparing the biased gene expression signature to the plurality of the instances, assigning a connectivity score to each of the plurality of instances, and formulating a skin care composition by mixing a dermatologically acceptable carrier with at least one skin care agent that is associated with an instance having a negative correlation (i.e., a negative connectivity score).

Measuring Gene Expression

The methods and systems herein utilize a gene expression measurement obtained from a biological sample. Some non-limiting examples of such biological samples include skin tissue samples obtained from human subjects (e.g., full thickness skin biopsies), primary cells (i.e., cultured cells isolated from human tissue), and cell lines (i.e., cultured cells that have been continually passaged over a long period of time and have acquired homogeneous genotypic and phenotypic characteristics). Gene expression can be detected and/or measured in a variety of ways such as, for example, using biomolecules representative of gene expression (“biomarkers”). Biomarkers commonly used in gene expression measurements include protein, nucleic acid, polynucleotides (e.g., microRNA, mRNA, and cDNA), protein fragments or metabolites, and/or products of enzymatic activity encoded by the protein encoded by a gene transcript. A particularly suitable biomarker for measuring gene expression is mRNA encoded by the genes of interest. In some embodiments, it may be desirable to reverse transcribe mRNA encoded by one or more genes of interest into cDNA and measure the cDNA. The mRNA or cDNA sample is measured by hybridizing the nucleic acids with oligonucleotides specific for mRNAs or cDNAs encoded by one or more of the genes of interest, optionally immobilized on a substrate (e.g., as an array or microarray), and measuring the binding level of the nucleic acid to the probe, e.g., using microarray reader to measure fluorescence produced by the hybridized biomarker. In some embodiments, it may be desirable to amplify the mRNA or cDNA, e.g., by polymerase chain reaction (PCR), prior to hybridization.

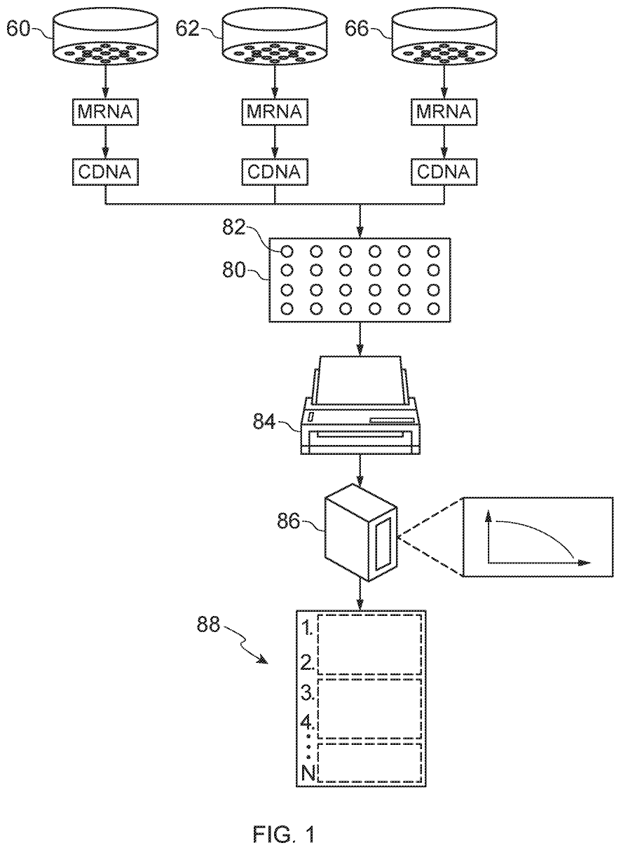

FIG. 1 illustrates an example of a method of constructing a gene expression profile from a skin tissue sample. The skin tissue sample may be obtained from a human donor by any suitable means known in the art (e.g., biopsy). In some embodiments, the skin tissue samples is separated into one or more components (e.g., suprabasal, basal, and dermal) via, e.g., laser capture micro-dissection to provide test samples 60 and 62. In this example, mRNA is extracted from the test samples 60 and 62 and a control sample 66, and then reverse transcribed to cDNA. The cDNA is marked with different fluorescent dyes (e.g., red and green), if a two-color microarray analysis is to be performed, or the cDNA may be prepped for a one-color microarray analysis. A plurality of replicates may be processed if desired. The cDNA is co-hybridized to a microarray 80 comprising a plurality of gene probes 82 (e.g., between 10,000 and 50,000). The microarray 80 is scanned by a scanner 84, which excites the fluorescent dyes and measures the amount of fluorescence. A computer 86, which may be integrated with the scanner or a standalone device, analyzes the fluorescence data to determine the expression levels of the genes. The expression levels may include: i) up-regulation (i.e., greater binding of the cDNA from the test sample (test cDNA) to the probe than the cDNA from the control sample (control cDNA); ii) down-regulation (i.e., greater binding of control cDNA to the probe than the test cDNA); iii) expressed but not differentially (i.e., similar binding of the control cDNA and the test cDNA); and iv) no detectable signal or noise. The up-regulated and down-regulated genes are referred to as differentially expressed. The differentially expressed genes may be ranked ordered and stored as a digital file 88.

Microarrays and conventional microarray analysis techniques are known, and it is contemplated that any suitable microarray technology and associated techniques may be used. For example, Affymetrix GeneChip™ technology and Illumina BeadChip™ technology may be particularly suitable for quantifying gene expression in the methods and systems herein.

Calculating a Gene Expression Consistency Value

One factor that can impact the quality of the gene expression signature is the number of genes included in the signature. With respect to a cosmetic data architecture and connectivity map, too few genes can result in a signature that is unstable for the highest scoring instances. In other words, small changes to the gene expression signature can result in significant differences in the highest scoring instance. Conversely, too many genes may tend to partially mask the dominant biological responses and will include a higher fraction of genes meeting statistical cutoffs by random chance—thereby adding undesirable noise to the signature. Thus, it may be desirable to apply a suitable statistical filter to a gene expression profile (e.g., p-values from a t-test, ANOVA, correlation coefficient, or other model-based analysis) to tailor the number of genes to a suitably sized gene expression signature. Limiting the gene expression signature to genes that meet some reasonable cutoff for statistical significance compared to an appropriate control is important to allow selection of genes that are characteristic of the skin condition of interest. Using a statistical method is preferable to using a fold change value, which does not take into account the noise around the measurements. The t-statistic may be particularly suitable for selecting the genes in a signature because it can indicate the directionality of the gene expression changes (i.e., up- or down-regulated) as well as statistical significance. As one example, p-values may be chosen as the statistical measure, and a cutoff value of p≤0.05 or p≤0.1 may be chosen. The resulting p-values may be used to represent the gene expression consistency values, which can be rank ordered as a list of identifiers representing genes that are differentially expressed. The ordered list of identifiers can be optionally associated with a numerical ranking for the identifier corresponding to its rank in the ordered list (e.g., 1 to N, where N is the number of genes in the list).

It is believed, without being limited by theory, that when a gene expression analysis of a skin condition yields between about 2,000 and 4,000 genes, having a statistical p-value of less than 0.05 and approximately 1000 genes having a p-value of less than 0.001, a very strong biological response is indicated. A moderately strong biological response may yield approximately 800-2000 genes have a statistical p-value of less than 0.05 combined with approximately 400-600 genes have a p-value of less than 0.001. In these cases, a gene expression signature comprising between 100 and 600 genes appears to be suitable for predicting putative skin care agents. Weaker biology may be better represented by a gene expression signature comprising fewer genes, such as between about 20 and 100 genes.

Constructing a Biased Gene Expression Signature

Conventional CMap techniques generally involve querying a database of instances with a gene expression signature constructed using gene expression profile data obtained from skin tissue that exhibits a skin condition of interest (“condition signature”). One challenge to this approach is ensuring that the genes used to construct the signature reflect the dominant and key biology for the skin condition of interest without including genes that are non-informative. It has now been discovered that biasing a condition signature with gene expression data from a benchmark signature can improve the predictability of a CMap query with regard to identifying potential skin care agents for treating the subject skin condition.

Constructing a biased gene expression signature generally involves filtering a condition signature through a benchmark signature to identify and/or remove genes from the condition signature that are not responsive to treatment by the benchmark agent or are modulated in opposite directions. Thus, filtering the condition signature through the benchmark signature will typically result in some of the top most up- or down-regulated genes to be removed from the condition signature and/or genes that would not normally be in the top up- or down-regulated genes to be added to the signature. The most significant genes remaining after filtering may then be used as the biased signature. In some instances, the biased gene expression signature may comprise the top 200 up-regulated genes and top 200 down-regulated genes (e.g., between 50 and 300 genes each or between 100 and 250 each).

The gene expression profile for a skin condition of interest may include between 1000 and 5000 significantly regulated genes, some or all of which are used to construct the condition signatures. The significantly regulated genes may or may not be distributed evenly between up-regulated genes and down-regulated genes. Each significantly regulated gene in the condition signature is filtered through a benchmark signature. In some embodiments, if the gene in the condition signature is not significantly regulated in the benchmark signature or if it is not significantly regulated in the opposite direction in the benchmark signature, then that gene is removed from the condition signature. In some embodiments, genes in the condition signature that are significantly regulated in the same direction in the benchmark signature may be included in the biased condition signature, if the direction of regulation corresponds to an improvement in the skin condition, based on the known function of the subject gene(s). The genes remaining in the condition signature after filtering through the benchmark signature may be used as the biased condition signature. However, it may be desirable to select, for example, only the top 50, 100, 150, 200, or 250 up-regulated and top 50, 100, 150, 200, or 250 down-regulated genes for use in the biased condition signature.

A benchmark signature may be constructed by contacting skin cells (e.g., keratinocytes or fibroblasts) with a benchmark skin agent (e.g., Tri-luma® brand skin cream) and measuring the level of differential gene expression relative to a control (i.e., skin cells of the same type that have not been treated with the benchmark skin agent). The type of skin cell selected to construct the benchmark signature depends on the cell type used to generate the relevant instances in the CMap database (i.e., the instances that will be queried with the biased condition signature). For example, if the biased condition signature will be used to query instances generated from keratinocytes, then keratinocytes (e.g., a commercially available human tert-keratinocytes cell line) are used to construct the benchmark signature. Alternatively, if the biased condition signature will be used to query instances generated from fibroblasts, then fibroblasts (e.g., human BJ Fibroblast cell line from ATCC, Manassas, Va.) are used to construct the benchmark signature. The resulting benchmark expression profile may be subjected to a suitable statistical analysis to identify the significantly up-regulated and down-regulated genes (e.g., p≤0.1 or p≤0.05) for use in constructing the benchmark signature. The significantly up- and down-regulated genes may be rank ordered, e.g., according to p-value, and stored as a digital file.

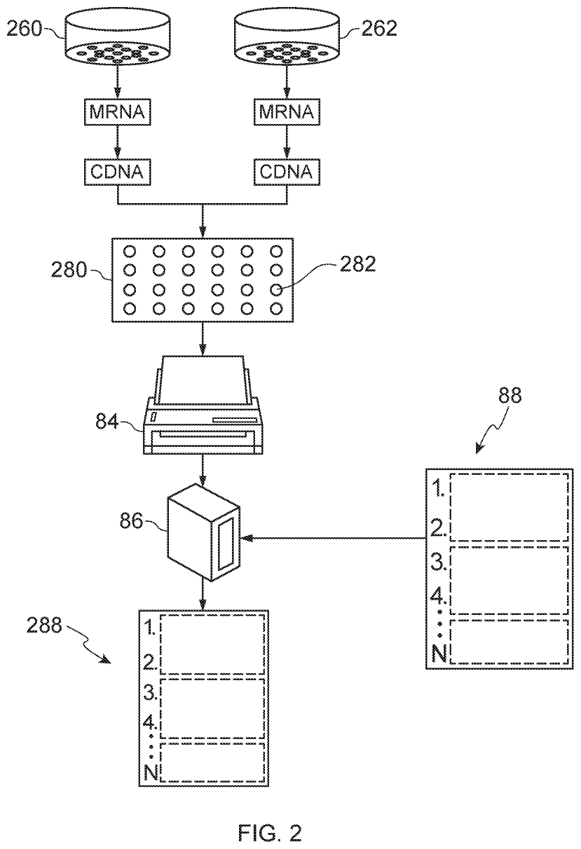

FIG. 2 illustrates an example of a method of constructing a biased gene expression signature 288. In this example, a benchmark signature is obtained by treating skin test cells 260 with a benchmark agent. In this example, mRNA is extracted from the treated skin test cells 260 and skin control cells 262 and reverse transcribed to cDNA. The cDNA is marked with different fluorescent dyes (e.g., red and green) if a two-color microarray analysis is to be performed, or the samples may be prepped for a one-color microarray analysis. A plurality of replicates may be processed if desired. The cDNA is co-hybridized to a microarray 280 comprising a plurality of gene probes 282. The microarray 80 is scanned by a scanner 84, which excites the fluorescent dyes and measures the amount of fluorescence. A computer 86, which may be integrated with the scanner or a standalone device, analyzes the fluorescence data to determine the expression levels of a gene. The expression levels can be the same as those for the condition signature described above with regard to FIG. 1. The condition signature 88 is filtered through the benchmark signature by the computer 86 to generate a biased condition signature 288.

Systems, Devices, and Computer-Related Aspects of the Invention

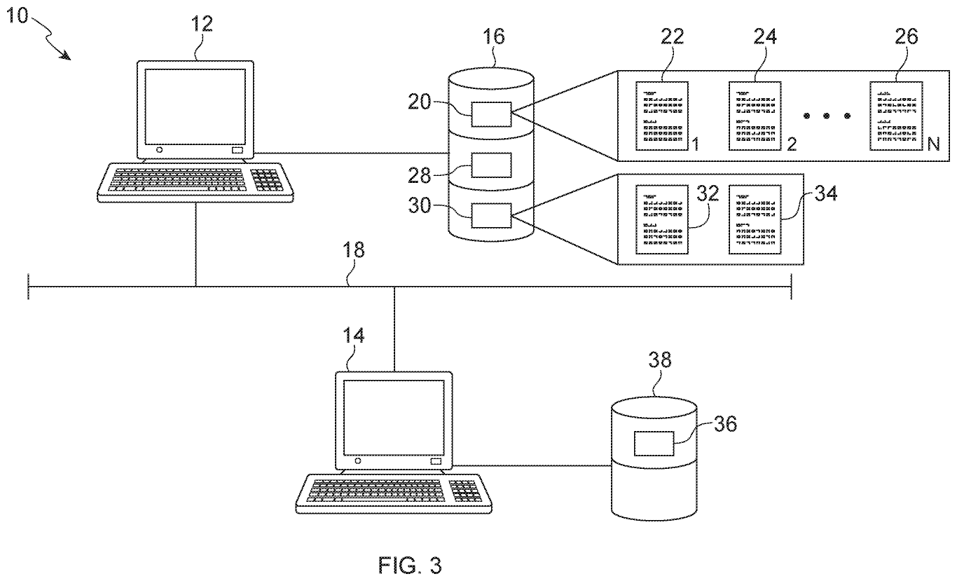

Various aspects of the present method employ the use of a computer and computer-based systems and devices. FIG. 3 illustrates an example of systems and devices that may be suitable for use with the method herein. As illustrated in FIG. 3, system 10 includes one or more computers 12, 14, a computer readable medium 16, and a communication network 18. The computer readable medium 16, which may be provided as a hard disk drive, comprises a digital file 20, such as a database file, comprising a plurality of instances 22, 24, and 26 stored in a data structure associated with the digital file 20. A plurality of instances may be stored in relational tables and indexes or in other types of computer readable media. The instances 22, 24, and 26 may also be distributed across a plurality of digital files, a single digital file 20 being described herein however for simplicity. The instances may be constructed according to the methods described in more detail below. The digital file 20 can be provided in wide variety of formats such as, for example, a word processing file format (e.g., Microsoft® Word®), a spreadsheet file format (e.g., Microsoft® Excel®), and/or a database file format. Some non-limiting examples of file formats include those associated with file extensions such as *.xls, *.xld, *.xlk, *.xll, *.xlt, *.xlxs, *.dif, *.db, *.dbf, *.accdb, *.mdb, *.mdf, *.cdb, *.fdb, *.csv, *.sql, *.xml, *.doc *.txt, *.rtf, *.log, *.docx, *.ans, *.pages, *.wps, etc.

Instances 22, 24, and 26 include an ordered listing of microarray probe set IDs, wherein the value of N is equal to the total number of probes on the microarray used in the analysis, which can exceed 20,000 for some microarrays. Some non-limiting examples of microarrays suitable for use herein include Affymetrix™ GeneChips™ brand microarrays (e.g., HG-U133 Plus 2.0, HG-U219, and HG-U133A2.0) and Illumina™ BeadChip™ brand microarrays. The ordered listing may be stored in a data structure of the digital file 20 and the data arranged so that, when the digital file is read by the software application 28, a plurality of character strings are reproduced representing the ordered listing of probe set IDs. While it is preferred that each instance comprise a full list of the probe set IDs, it is contemplated that one or more of the instances may comprise less than all of the probe set IDs of a microarray. It is also contemplated that the instances may include other data in addition to or in place of the ordered listing of probe set IDs. For example, an ordered listing of equivalent gene names and/or gene symbols may be substituted for the ordered listing of probe set IDs. Additional data may be stored with an instance and/or the digital file 20. In some embodiments, the additional data is referred to as metadata and can include one or more of cell line identification, batch number, exposure duration, and other empirical data, as well as any other descriptive material associated with an instance ID. The ordered list may also comprise a numeric value associated with each identifier that represents the ranked position of that identifier in the ordered list.

As illustrated in FIG. 3, the computer readable medium 16 may also have a second digital file 30 stored thereon. The second digital file 30 may include one or more lists 32, 34 of microarray probe set IDs associated with one or more gene expression profiles, condition signatures, benchmark signatures, and/or biased condition signatures. The listing(s) may be stored in a data structure of the digital file 30 and the data arranged so that, when the digital file is read by the software application 28, a plurality of character strings are reproduced representing the list of probe set IDs. Instead of probe set IDs, equivalent gene names and/or gene symbols (or another nomenclature) may be substituted for a list of probe set IDs. Additional data may be stored with the gene expression data and/or the digital file 30 and this is commonly referred to as metadata, which may include any associated information, for example, cell line or sample source and micro array identification.

The data stored in the first and/or second digital files 20 and/or 30 may be stored in one or more searchable databases, which can be accessed by a user of the system 10 (e.g., via a graphical user interface associated with a database management system) to access and retrieve the desired data. The digital files 20, 30 may include data that is transmitted across the communication network 18 from a digital file 36 stored on the computer readable medium 38 associated with a second computer 14. In some embodiments, the computer readable medium 16 includes a digital file 28 with computer readable instructions or software for reading, writing to, or otherwise managing and/or accessing the digital files 20, 30. The computer readable medium 16 may also include software or computer readable and/or executable instructions that cause the computing device 12 to perform one or more steps of the methods described herein, including, for example, the step(s) associated with comparing a gene expression signature stored in digital file 30 to instances 22, 24, and 26 stored in digital file 20. In some embodiments, the one or more digital files 28 may form part of a database management system for managing the digital files 20, 30. Non-limiting examples of database management systems are described in U.S. Pat. Nos. 4,967,341 and 5,297,279.

The computer readable medium 16 may form part of or otherwise be connected to the computer 12. The computers 12, 14 of the system 10 may operate in a networked environment across network 18 using a wired and/or wireless network communications interface. For example, the communication network 18 may be a wide area network (WAN) such as the Internet, or a local area network (LAN). The communication network 18 may include any necessary hardware such as, for example, base stations for wireless communications, which include transceivers, associated electronic devices for modulation/demodulation, and switches and ports to connect to a network.

Identifying Potential Skin Care Agents and Formulating Skin Care Compositions

The biased condition signature described herein is useful for its improved ability, relative to an unbiased condition signature, to identify connections between a perturbagen and the ability of the perturbagen to modulate genes associated with a skin condition. For example, the biased condition signature can be used to identify skin care agents that can potentially improve the appearance of a skin pigmentation condition. Indeed, the present method lends itself to an improved method of screening large libraries of candidate skin care agents in silico to identify lead candidates for further evaluation using, e.g., the in vitro and ex vivo methods described herein.

The present method comprises querying a data architecture of stored skin instances with a biased gene expression signature. Each skin instance is associated with a skin care agent. In the query, the biased condition signature is compared to each stored instance (i.e., each gene identifier in the biased condition signature list is compared with the position of the same identifier in each instance list). Comparing the biased condition signature to each stored instance includes assigning a connectivity score to each of the instances. In some embodiments, a skin care agent associated with an instance may be identified as a candidate for treating the skin condition of interest (i.e., the skin condition associated with the biased condition signature) when the instance has a negative connectivity score (which represents a negative correlation between the biased condition signature and instance) or a positive connectivity score (which represents a positive correlation between the biased condition signature and instance), depending on the functions of the genes being regulated. For example, if the connectivity score corresponds to a change in gene expression that indicates an improvement in the skin condition, then the skin care agent may be a candidate for further testing and/or incorporation into a skin care composition.

Skin care compositions herein may be made by combining a skin care agent with a dermatologically acceptable carrier using materials, processes, and equipment known to those skilled in art for making such compositions. Such methods typically involve mixing of the ingredients in one or more steps to a relatively uniform state, with or without heating, cooling, application of vacuum, and the like. The compositions are preferably prepared to optimize stability (physical stability, chemical stability, photostability) and/or delivery of the active materials (e.g., hexamidine, sugar amine, vitamin B₃, retinyl propionate, phytosterol). This optimization may include appropriate pH (e.g., less than 7), exclusion of materials that can complex with the skin care agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen, sunblock, use of opaque packaging), etc.

The skin care compositions herein may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some non-limiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

Generating Instances, Ordering Data

In some embodiments, the inventive method involves querying a data architecture of instances. Each instance comprises or consists essentially of transcriptional data obtained from a gene expression profiling experiment, wherein a biological sample is exposed to a perturbagen. For example, each instance may include the rank ordered transcriptional data for all the probe sets on an Affymetrix HG-U219 GeneChip, wherein each probe on the microarray has a unique probe set IDentifier. The probe sets can be rank ordered by the fold-change level of gene expression detected relative to controls in the same CMap batch (single instance/average of controls). It may be desirable to rank order the probe set identifiers to reflect the most up-regulated to the most down-regulated. Suitable methods for generating instances, including methods of thresholding to reduce noise in the signal values, are disclosed in U.S. Pat. No. 9,434,993 to Binder, et al. and U.S. Publication Nos. 2013/0261007 and 2017/0343534.

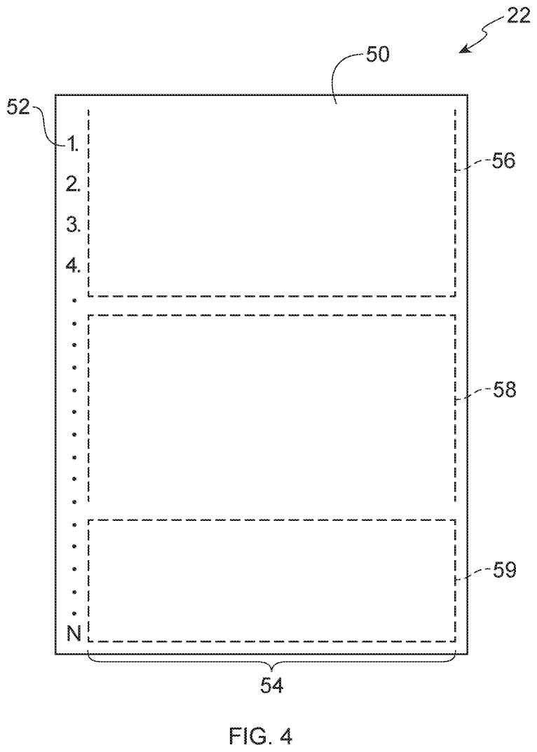

The rank ordered data may be stored as an instance or a gene expression profile. FIG. 4 illustrates an example of an instance 22. The probe IDs may be sorted into a rank ordered list 54 according to the level of gene expression regulation detected, wherein the list progresses from up-regulated to marginal or no regulation to down-regulated. As illustrated in FIG. 4, the data associated with the instance 22 (or a gene expression profile associated with a skin condition) comprises a probe ID 50 and a value 52 representing its ranking in the list 54 (e.g., 1, 2, 3, 4 . . . N, where N represents the total number of probes on the microarray). The ordered list 54 may generally comprise approximately three groupings of probe IDs: a first grouping 56 of probe IDs associated with up-regulated genes, a second group 58 of probe IDs associated with genes with marginal regulation or no detectable signal or noise, and a third group 59 of probe IDs associated with down-regulated genes. The most up-regulated genes are at or near the top of the list 54 and the most down-regulated genes are at or near the bottom of the list 54. The groupings are shown for illustration, but the lists for each instance 22 may be continuous and the number of regulated genes will depend on, e.g., the strength of the effect of the perturbagen associated with the instance 22. Other arrangements within the list 54 may be provided. For example, the probe IDs associated with the down-regulated genes may be arranged at the top of the list 54. This data in the instance 22 may also further comprise metadata such as skin care agent identification and/or concentration, cell line or sample source, and microarray identification.

Comparing Biased Gene Expression Signature(S) to Instances

Broadly, the present method includes querying a data architecture of stored instances with a biased condition signature and applying a statistical method to determine how strongly the biased gene expression signature genes match the regulated genes in an instance. Positive connectivity occurs when the genes in the up-regulated gene expression signature list are enriched among the up-regulated genes in an instance and the genes in the down-regulated gene expression signature list are enriched among the down-regulated genes in an instance. On the other hand, if the up-regulated genes of the gene expression signature are predominantly found among the down-regulated genes of the instance, and vice versa, this is scored as negative connectivity. The resulting rank ordered list of instances may be displayed to a user using any suitable software and computer hardware allowing for visualization of data.

The connectivity score is a combination of an up-score and a down-score, wherein the up-score represents the correlation between the up-regulated genes of a gene expression signature and an instance and the down-score represents the correlation between the down-regulated genes of a gene expression signature and an instance. The up-score can be calculated by comparing each identifier of an “up list” of a biased gene expression signature (i.e., the list that contains the up regulated genes of the signature), to an ordered instance list, and the down-score can be calculated by comparing each identifier of a “down list” of a gene signature (i.e., the list that contains the down regulated genes of the signature) to an ordered instance list. The sign of the connectivity score is determined by whether the instance links positivity or negatively to the gene expression signature. Positive connectivity occurs when a perturbagen associated with an instance tends to up-regulate the genes in the up list of the signature and down-regulate the genes in the down list. Conversely, negative connectivity occurs when the perturbagen tends to reverse the up- and down-signature gene expression changes. The magnitude of the connectivity score is the sum of the absolute values of the up and down scores when the up and down scores have different signs. A high positive connectivity score predicts that the perturbagen will tend to induce the condition associated with the query gene expression signature, and a high negative connectivity score predicts that the perturbagen will tend to reverse the condition associated with the query gene expression signature. A zero score is assigned where the up- and down-scores have the same sign, indicating that a perturbagen would not be predicted to have a consistent impact on a skin condition.

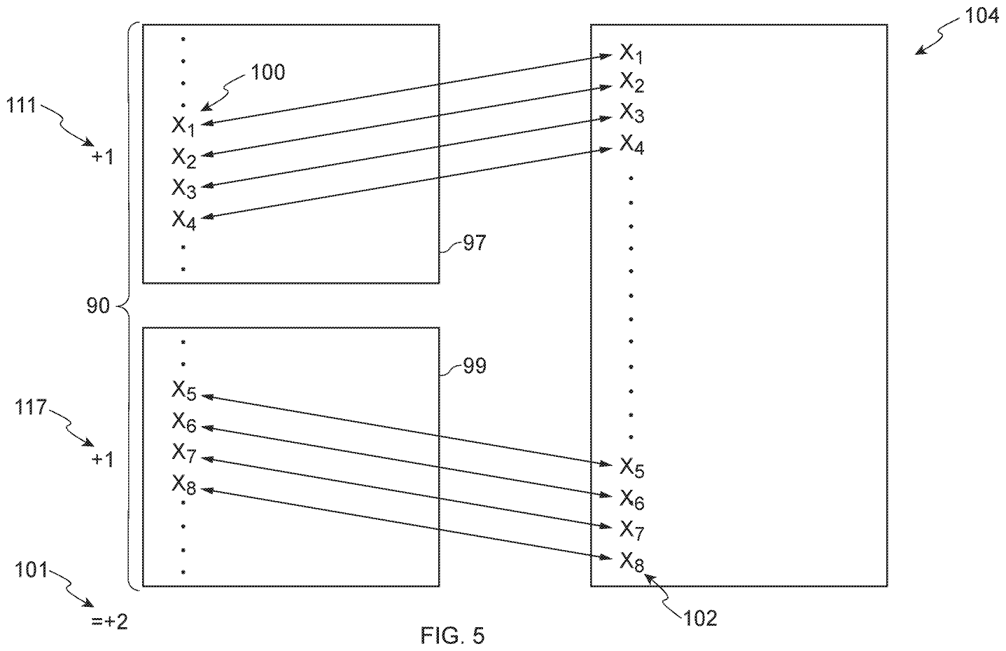

FIG. 5 illustrates an extreme example of positive connectivity between a biased condition signature 90 and an instance 104, wherein the probe IDs 102 of the instance 104 are ordered from most up-regulated to most down-regulated. In this example, the probe IDs 100 (e.g., X₁, X₂ X₃, X₄, X₅, X₆, X₇, X₈) of the biased condition signature 90, are arranged as an up list 97 and a down list 99. As illustrated in FIG. 5, the probe IDs 100 in the up list 97 of the biased condition signature 90 have a one-to-one positive correspondence with the most up-regulated probe IDs 102 of the instance 104, and the probe IDs 100 in the down list of the biased condition signature 90 have a one-to-one positive correspondence with the most up-regulated probe IDs 102 of the instance 104.

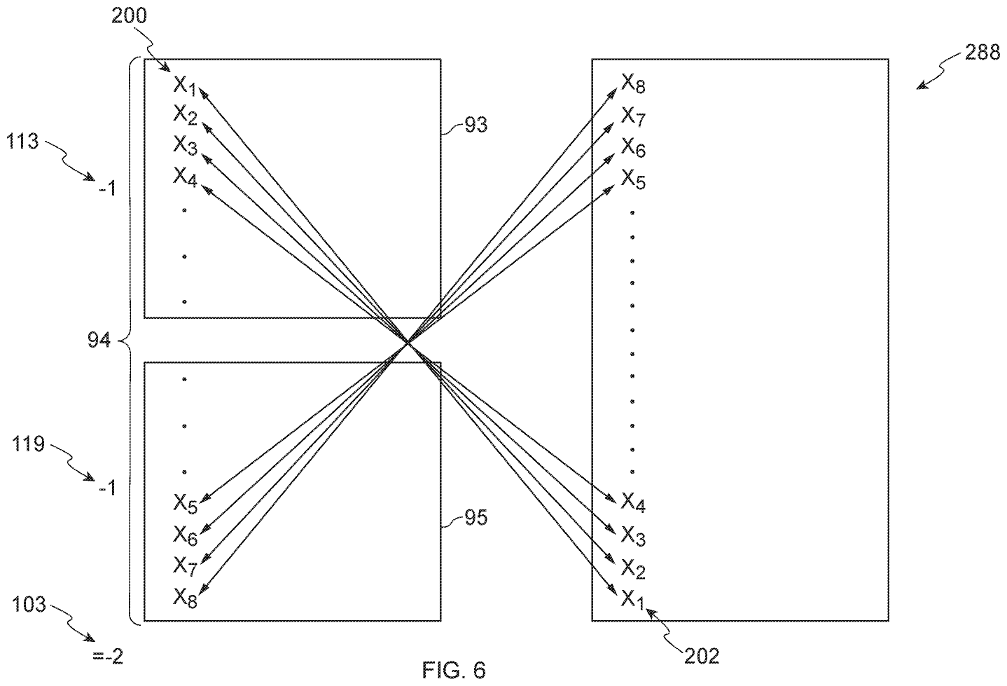

FIG. 6 illustrates an extreme example of negative connectivity between a biased condition signature 94 and an instance 288. The probe IDs 202 of the instance 288 are ordered from most up-regulated to most down-regulated. In this example, the probe IDs 200 of the up list 93 (e.g., X₁, X₂ X₃, X₄) of the biased condition signature 94 correspond exactly with the most down-regulated genes of the instance 288, and the probe IDs 200 of the down list 95 (e.g., X₅, X₆, X₇, X₈) correspond exactly to the most up-regulated probe IDs 202 of the instance 288.

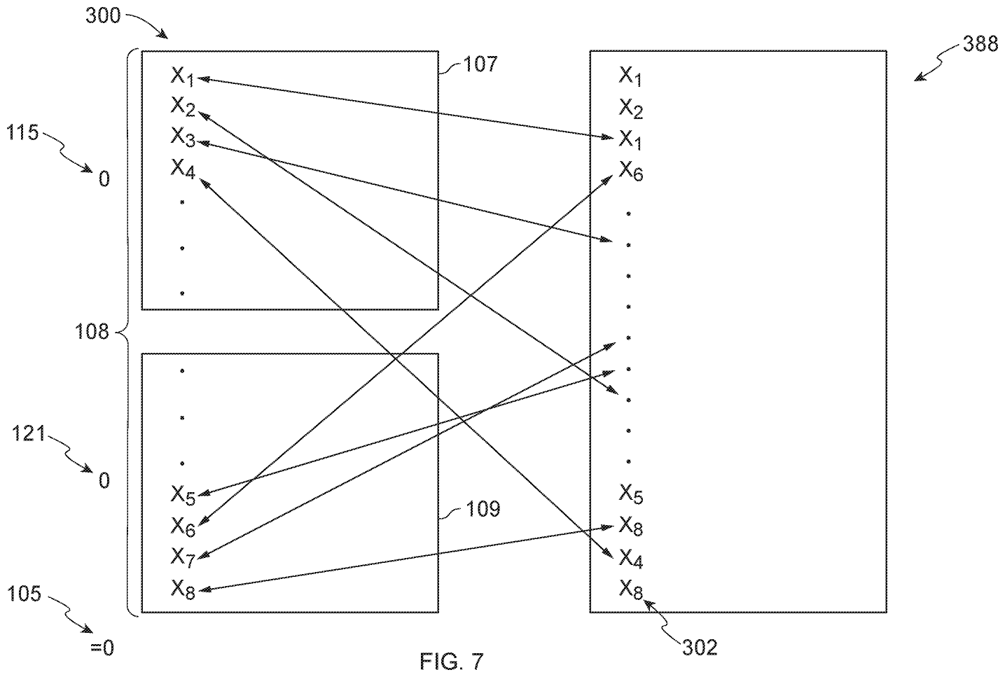

FIG. 7 illustrates an extreme example of neutral connectivity, wherein there is no consistent enrichment of the up- and down-regulated genes of the biased condition signature 108 among the up- and down-regulated genes of the instance 388, either positive or negative. Hence the probe IDs 300 (e.g., X₁, X₂ X₃, X₄, X₅, X₆, X₇, X₈) of the biased gene expression signature 108 (comprising an up list 107 and a down list 109) are scattered with respect to rank with the probe IDs 302 of the instance 388.

As illustrated in FIGS. 5, 6, and 7, the value of the connectivity score 101, 103, and 105 may range from +2 (greatest positive connectivity) to −2 (greatest negative connectivity), and are a combination of the up score 111, 113, 115 and the down score 117, 119, 121. The strength of matching between a gene expression signature and an instance represented by the up scores and down scores and/or the connectivity score may be derived by one or more approaches known in the art and include, but are not limited to, parametric and non-parametric approaches. Examples of parametric approaches include Pearson correlation (or Pearson r) and cosine correlation. Examples of non-parametric approaches include Spearman's Rank (or rank-order) correlation, Kendall's Tau correlation, and the Gamma statistic. Optionally, in order to eliminate a requirement that all profiles be generated on the same microarray platform, a non-parametric, rank-based pattern matching strategy based on the Kolmogorov-Smirnov statistic (see M. Hollander et al. “Nonparametric Statistical Methods”; Wiley, New York, ed. 2, 1999) (see, e.g., pp. 178-185) is used. Where all expression profiles are derived from a single technology platform, similar results may be obtained using conventional measures of correlation, for example, the Pearson correlation coefficient. It may be desirable to use a rank-based pattern-matching strategy based on the Kolmogorov-Smirnov statistic, which has been refined for gene profiling data and is known as Gene Set Enrichment Analysis (GSEA) (see, e.g., Lamb et al. 2006 and Subramanian, A. et al. (2005) Proc. Natl. Acad Sci U.S.A., 102, 15545-15550).

The displayed rank-ordered list of instances may be used to identify (i) one or more perturbagens associated with the instances of interest (thereby correlating activation or inhibition of a plurality of genes listed in the query signature to one or more potential skin care agents); (ii) differentially expressed genes associated with any instances of interest (thereby correlating such genes with the one or more perturbagens, the skin condition of interest, or both); (iii) the cells associated with any instance of interest (thereby correlating such cells with one or more of the differentially expressed genes, the one or more perturbagens, and the skin condition of interest); or (iv) combinations thereof. The perturbagen(s) associated with an instance may be identified from the metadata stored in the database for that instance. However, one of skill in the art will appreciate that skin care agent data for an instance may be retrievably stored in and by other means. Because the identified skin care agents statistically correlate to activation or inhibition of genes listed in the query signature, and because the query signature is a proxy for a skin condition, the identified perturbagens may be candidates for new skin care agents, new uses of known skin care agents, or to validate known skin care agents for known uses.

Characterizing Perturbagen Activity in Models of Skin Conditions

In some embodiments, the methods herein include characterizing the activity of a perturbagen associated with an instance (i.e., a candidate skin care agent) in one or more assays to evaluate its potential usefulness as a skin care agent. For example, a perturbagen identified as a potential skin care agent as a result of querying a database of instances with a biased gene expression signature may be further subjected to in vitro testing (e.g., cell-based assays or ex vivo tissue assays) and/or in vivo assays involving human participants (e.g., clinical studies) to evaluate or validate its efficacy in treating a skin condition of interest. In some embodiments, a tiered screening method may be used, wherein a potential skin care agent is identified using the improved CMap technique herein and then the activity of the identified agent is characterized in a cell-based assay, an ex vivo tissue assay, and/or in vivo. Some non-limiting examples of assays that may be used for validating the efficacy of a potential skin care agent (e.g., as part of a tiered screening method) include phenotypic assays in which melanin content is quantitated as an endpoint, such as a B16 Mouse Melanoma melanin synthesis assay, a reconstructed skin model with a melanocyte component (e.g., SkinEthic™ from EPISKIN), and/or a skin explant assay (e.g., CuTech™). It may be desirable to select validation assays that cover mechanistic space exhibited by the benchmark material. For example, hydroquinone is known to inhibit melanin synthesis, so it may be desirable to configure or select a validation that has a melanin production component. By combining the improved CMap technique herein with confirmatory in vitro and/or in vivo testing, a coherent, tiered system of assays can be provided for characterizing the influence of a potential skin care agent on a skin condition of interest. In various embodiments, the activity of a perturbagen is compared to a benchmark. An exemplary benchmark for a cell proliferation assay and an inflammation assay is clobetasol, a corticosteroid used to treat, e.g., eczema and psoriasis.

Skin Care Compositions and Methods of Use

Skin care compositions comprising an effective amount of skin care agent identified according to the methods herein can be used to regulate a skin condition by topically applying the skin care composition to a target portion of skin in need of regulation. The present method may include identifying a target portion of skin in need of treatment and/or where treatment is desired, and applying the skin care composition to the target portion of keratinous tissue during a treatment period. Identifying a target portion of keratinous tissue in need of treatment can be based on the presence of a visible condition (e.g., hyperpigmentation, uneven skin tone, or wrinkles). In some instances, the target portion of skin may not exhibit visible signs of a condition, but a user may still wish to target such an area if it is one that is known to develop a condition (e.g., skin surfaces that are typically not covered by clothing).

Suitable treatment periods include daily application, twice daily application, or an even more frequent daily basis for a sufficient time for the skin care agent to provide the desired benefit. For example, the treatment period may be of sufficient time for the skin care agent to provide a noticeable and/or measurable improvement in the skin condition. The treatment period may last for at least 1 week (e.g., about 2 weeks, 4 weeks, 8 weeks, or even 12 weeks). In some instances, the treatment period will extend over multiple months (i.e., 3-12 months) or multiple years. In some instances, a skin care composition containing an effective amount of a skin care agent may be applied most days of the week (e.g., at least 4, 5 or 6 days a week), at least once a day or even twice a day during a treatment period of at least 2 weeks, 4 weeks, 8 weeks, or 12 weeks.

The skin care compositions herein may be applied locally or generally. In reference to application of the composition, the terms “localized”, “local”, or “locally” mean that the composition is delivered to the targeted area while minimizing delivery to keratinous surfaces where treatment is not desired. While certain embodiments herein contemplate applying a composition locally to an area, it will be appreciated that the compositions herein can be applied more generally or broadly to one or more keratinous surfaces. In certain embodiments, the compositions herein may be used as part of a multi-step beauty regimen, wherein the present composition may be applied before and/or after one or more other compositions.

The skin care compositions may be applied by any suitable means known for applying such products, including rubbing, wiping or dabbing with hands, fingers and/or an implement. Non-limiting examples of implements include a sponge or sponge-tipped applicator, a swab (for example, a cotton-tipped swab), a pen optionally comprising a foam or sponge applicator, a brush, a wipe, and combinations thereof. The composition may be pre-applied to the applicator and, for example, delivered to the user pre-packaged as such, or the user may be instructed to apply the composition to the applicator prior to use. In some instances, the composition may be stored in an implement, for example, in a separate storage area for the composition. In this example, the composition may be transferred to the applicator from the storage area, for example, by squeezing and/or breaking or by other suitable means. The composition may be applied to the keratinous tissue by contacting the applicator and composition to the skin. Contact may include, for example, light pressure, dabbing, rubbing, wiping, or any other suitable means.

The skin care compositions herein include a dermatologically acceptable carrier and, optionally, other ingredients commonly included in cosmetic skin care compositions. The carrier may be present at an amount of 20% to 99.99% (e.g., 50% to 99%, 60% to 98%, 70% to 95%, or even 60% to 80%) by weight of the composition. The carrier may be aqueous or anhydrous. The form of the carrier is not particularly limited, and can be any suitable form known in the art for the application desired (e.g., solutions, dispersions, emulsions and combinations thereof). “Emulsions” refer to compositions having an aqueous phase and an oil phase. Emulsion carriers include, but are not limited to oil-in-water, water-in-oil and water-in-oil-in-water emulsions. Emulsion carriers herein may include from 0.01% to 10% (e.g., 0.1% to 5%) of an emulsifier (e.g., nonionic, anionic, cationic emulsifier, or a combination thereof). Suitable emulsifiers are disclosed in, for example, U.S. Pat. Nos. 3,755,560, 4,421,769, and McCutcheon's Detergents and Emulsifiers, North American Edition, pages 317 324 (1986).

The compositions of the present invention may contain a variety of optional ingredients that are conventionally used in skin care compositions, as long as the optional ingredient(s) do not undesirably alter product stability, aesthetics or performance. The optional ingredients, when incorporated into the composition, should be suitable for contact with human skin without undue toxicity, incompatibility, instability, allergic response, and the like within the scope of sound judgment. The CTFA Cosmetic Ingredient Handbook, Second Edition (1992) describes a wide variety of non-limiting cosmetic and pharmaceutical ingredients. The compositions herein may include 0.0001% to 50%; 0.001% to 20%; or, even 0.01% to 10%, by weight of the composition, of optional ingredients. Some non-limiting examples of optional ingredients include vitamins, minerals, peptides and peptide derivatives, sugar amines, oil control agents, flavonoid compounds, anti-oxidants and/or anti-oxidant precursors, preservatives, phytosterols, protease inhibitors, tyrosinase inhibitors, anti-inflammatory agents, moisturizing agents, emollients, humectants, exfoliating agents, skin lightening agents, sunscreens, sunless tanning agents, pigments, film formers, thickeners, pH adjusters, opacifying agents, colorings/colorants, particles, fragrances, essential oils, lubricants, anti-acne actives, anti-cellulite actives, chelating agents, anti-wrinkle actives, anti-atrophy actives, phytosterols and/or plant hormones, N-acyl amino acid compounds, antimicrobials, antifungals, and combinations of these. Other non-limiting examples of skin conditioning agents can be found in U.S. Pub. Nos. 2010/0272667 and 2008/0206373 and U.S. Pat. No. 8,790,720.

### Example

The following example illustrates a method of constructing a biased gene expression signature for a skin hyperpigmentation condition. The biased gene expression provided in this example can be used to query a database of instances to identify potential skin care actives useful for treating a skin pigmentation condition.

### Sample Collection and Processing

Skin tissue samples for use as test samples or control samples were collected from 77 human female donors aged 20 to 70 via a 2 mm punch biopsy. The test samples in this example were collected from a hyperpigmented spot present on facial skin. Each hyperpigmented spot that was sampled was visually classified by a dermatologist as one of the following hyperpigmentation conditions: solar lentigo, seborrheic keratosis, melasma, freckles, resolving acne lesions, and post-inflammatory hyperpigmentation. The control samples were collected from an adjacent portion of skin that did not include the hyperpigmentation condition (i.e., did not include a hyperpigmented spot). The skin tissue samples (i.e., test samples and control samples) were separated into three compartments (suprabasal, basal, and dermal) using LCM. The dermal compartment was subjected to further LCM to remove the hair follicles. The suprabasal compartment typically contains about 98% keratinocytes. The basal compartment typically contains about 90% keratinocytes and about 10% melanocytes. And the dermal compartment typically contains about 70% fibroblasts after the hair follicles are removed. The suprabasal layer and the dermal layer were used as test samples due to the predominance of keratinocytes and fibroblasts present in these compartments, respectively.

### Generating the Condition Profiles and Control Profiles

Cells from the suprabasal compartment of the test samples and control samples were placed in separate containers (12-well plates) and lysed with 350 ul/well of RLT buffer (available from Qiagen). The cells were then transferred to a suitable 96-well plate, and stored at −20° C. RNA was isolated from the RLT buffer using Agencourt® RNAdvance® Tissue-Bind magnetic beads (available from Beckman Coulter) according to manufacturer's instructions. 0.25 ug of total RNA per sample was labeled using Affymetrix GeneChip® IVT Plus Reagent Kit (available from Life Technologies) according to the manufacturer's instructions. The resultant biotin labeled and fragmented cRNA was hybridized to Affymetrix U219 GeneChips® brand microarrays. Washing, staining, and scanning of the microarray is done using GeneTitan® according to Affymetrix instructions. These steps were repeated for the dermal compartment of the test samples as well as the suprabasal and dermal compartments of the control samples.

### Constructing the Condition Signatures

The gene expression profiles obtained from the microarray analysis of the suprabasal and dermal compartments of the test samples and control samples, as described above, were used to construct condition signatures. The condition signatures were developed by identifying the significant differentially expressed genes between a test sample and its corresponding control sample (p≤0.05) and rank ordering the up-regulated and down-regulated genes according to p-value. The number of significantly regulated genes included in each condition signature from the suprabasal and dermal compartments are shown in Table 1.

TABLE 1
Condition Signatures

| | | # Significantly | # Significantly |
|---|---|---|---|
| Hyperpigmentation | | Up- | Down- |
| Condition | Compartment | regulated genes | regulated genes |
| Freckle | Suprabasal | 1043 | 994 |
| | Dermal | 640 | 898 |
| Melasma | Suprabasal | 1674 | 1546 |
| | Dermal | 347 | 481 |
| Post-inflammatory | Suprabasal | 2129 | 3522 |
| Hyperpigmentation | Dermal | 2006 | 2661 |
| Resolving Acne | Suprabasal | 3029 | 4007 |
| Lesion | Dermal | 2507 | 3608 |
| Seborrheic Keratosis | Suprabasal | 2493 | 2064 |
| | Dermal | 1130 | 1469 |
| Solar Lentigo (type I) | Suprabasal | 1087 | 1670 |
| | Dermal | 262 | 344 |
| Solar Lentigo (type II) | Suprabasal | 1809 | 2132 |
| | Dermal | 332 | 586 |

### Constructing the Benchmark Signatures

Human telomerized keratinocytes (tKC) (available from University of Texas, Southwestern Medical Center, Dallas, Tex.) were grown in EpiLife® media with 1X Human Keratinocyte Growth Supplement (Invitrogen, Carlsbad, Calif.) on collagen I coated cell culture flasks and plates (Becton Dickinson, Franklin Lakes, N.J.). The resulting cultured keratinocytes were seeded into 6-well plates at approximately 20,000 cells/cm² 24 hours before exposure to the benchmark skin agent. Human skin fibroblasts (BJ cell line from ATCC, Manassas, Va.) were grown in Eagle's Minimal Essential Medium (ATCC) supplemented with 10% fetal bovine serum (HyClone, Logan, Utah) in normal cell culture flasks and plates (Corning, Lowell, Mass.). The cultured BJ fibroblasts were seeded into 6-well plates at 12,000 cells/cm² 24 hours before exposure to the benchmark agent. The number of cells seeded in the well-plates should be sufficient to yield 2-4 μg total RNA per well.

All cells were incubated at 37° C. in a humidified incubator with 5% CO₂. At time=−24 hours, the cells were trypsinized from T-75 flasks and plated into 6-well plates in basal growth medium. At t=0, the media was removed and replaced with a dosing solution. The dosing solution for test cells (i.e., cells exposed to the benchmark skin care agent) included Tri-luma® brand skin cream at a concentration of 0.01% (for the keratinocytes) and 0.1% (for the fibroblasts), by weight of the dosing solution. Dosing solutions for the control cells were the same as the test cells, except without the benchmark skin agent. After 6 hours of exposure to the dosing solutions, cells were lysed with 350 ul/well of RLT buffer containing β-mercaptoethanol (Qiagen, Valencia, Calif.), transferred to a 96-well plate, and stored at −20° C.

RNA from cell culture batches was isolated from the RLT buffer and gene expression profiles for the treated cells and the control cells were generated in the same way as described above for the condition signatures. The benchmark profile is compared to the control profile to identify significant differentially expressed genes (p≤0.05), which are rank ordered to provide the benchmark signature.

### Constructing the Biased Condition Signature

Each gene in the condition signature was filtered through the benchmark signature to construct the biased condition signature. If a gene present in the condition signature was not present in the benchmark signature (i.e., was not expressed or significantly regulated) or was not significantly regulated in the opposite direction, then that gene was removed from the condition signature. Once all the genes from the condition signature were filtered through the benchmark signature, between 50 and 100 of the top up-regulated and between 50 and 100 of the top down-regulated genes were selected for use as the biased condition signature. The number of genes used in each biased signature are shown in Table 2. Of course, it is to be appreciated that any number of up-regulated and/or down-regulated genes may be selected for use as the biased condition signature. It is believed, without being limited by theory, that querying a database of instances with the biased gene expression signature herein will yield a list of actives that are more likely to treat a hyperpigmented skin condition.

TABLE 2
Biased Condition Signatures

| | | # Significantly | # Significantly |
|---|---|---|---|
| Hyperpigmentation | | Up- | Down- |
| Condition | Compartment | regulated genes | regulated genes |
| Freckle | Suprabasal | 76 | 90 |
| | Dermal | 87 | 100 |
| Malasma | Suprabasal | 100 | 100 |
| | Dermal | 79 | 85 |
| Post-inflammatory | Suprabasal | 100 | 100 |
| Hyperpigmentation | Dermal | 100 | 100 |
| Resolving Acne | Suprabasal | 100 | 100 |
| Lesion | Dermal | 100 | 100 |
| Seborrheic Keratosis | Suprabasal | 100 | 100 |
| | Dermal | 100 | 100 |
| Solar Lentigo (type I) | Suprabasal | 100 | 100 |
| | Dermal | 53 | 49 |
| Solar Lentigo (type II) | Suprabasal | 100 | 100 |
| | Dermal | 47 | 100 |

### Query

A CMap database of 2658 materials was queried with a condition signature and a corresponding biased condition signature. The results for pterostilbene, a known skin lightening agent, are shown in Table 3 below. The CMap score used in this example has a range of +1 to −1, where +1 is the highest possible positive connectivity and −1 is the highest possible negative connectivity.

TABLE 3
Comparison of CMap Scores for Potential Skin Care Agent

| | Pterostilbene¹ | | Pterostilbene² | |
|---|---|---|---|---|
| | Condition | Biased | Condition | Biased |
| Skin Condition | Signature | Signature | Signature | Signature |
| Freckles | −0.13 | −0.80 | 0.15 | −0.75 |
| Melasma | 0 | −0.70 | 0.38 | −0.63 |
| Post Inflammation | 0 | −0.71 | 0 | −0.67 |
| Hyperpigmentation | | | | |
| Resolving Acne | 0 | −0.60 | 0.20 | −0.59 |
| Seborrheic Keratosis | −0.22 | −0.81 | −0.02 | −0.77 |
| Solar Lentigo type 1 | 0 | −0.75 | 0 | −0.69 |
| Solar Lentigo type 2 | 0 | −0.63 | 0.21 | −0.58 |

¹Pterowhite ® from Sabinsa Corp, New Jersey.
²Pteropure ® from ChromaDex, Inc., California.

As can be seen in Table 3, the biased condition signature resulted in a much higher negative correlation to the instance than the unbiased condition signature. Generally, if the absolute value of the CMap score is less than 0.4, it is not considered a potential skin care agent of interest, and will not be selected for the next round of testing (e.g., in vitro testing or in vivo clinical testing). In this example, using the conventional signature would not have identified pterostilbene as a potential agent for treating any of the listed skin conditions, whereas the results of the biased signature suggest that pterostilbene may be a potential agent for all the listed conditions.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An improved connectivity mapping method for identifying potential skin care agents, comprising:
a) constructing a condition signature from a skin tissue sample;
b) constructing a benchmark signature for a type of cell present in the skin tissue sample;
c) filtering the condition signature through the benchmark signature to provide a biased condition signature, wherein the biased condition signature consists of a plurality of up-regulated genes and a plurality of down-regulated genes that all have a p-value of 0.1 or less;
d) querying a database of instances with the biased condition signature, wherein each instance is associated with a skin care agent;
e) generating a connectivity score for each instance; and
f) identifying a skin care agent associated with an instance in the database as a potential skin care agent when the connectivity score of the instance has a negative correlation to the biased condition signature.

2. The method of claim 1, wherein constructing the condition signature comprises:
a) obtaining a human skin tissue sample from a portion of skin that exhibits a skin condition of interest;
b) obtaining a human skin tissue sample from the donor that does not exhibit the skin condition;
c) constructing a gene expression profile from each of the skin samples in (a) and (b), wherein the gene expression profiles each comprise a list of identifiers representing the genes in that gene expression profile;
d) comparing the gene expression profiles in (c) to one another to identify genes that are differentially expressed;
e) rank ordering a list of identifiers representing the differentially expressed genes; and
f) selecting a plurality of up-regulated genes and plurality of down-regulated genes from the rank ordered list of identifiers to provide the condition signature.

3. The method of claim 1, wherein constructing the benchmark signature comprises:
a) exposing a plurality of cells to a benchmark skin agent, wherein the plurality of cells are of the type for which the benchmark signature is desired;
b) constructing a gene expression profile from the plurality of cells;
c) comparing the gene expression profile from the cells contacted with the benchmark skin care agent to a control profile to identify genes that are differentially expressed; and
d) rank ordering a list of identifiers representing the differentially expressed genes to provide the benchmark signature.

4. The method of claim 1, wherein the type of cell is selected from the group consisting of keratinocytes, fibroblasts, melanocytes, and melanoma cells.

5. The method of claim 1, wherein the skin condition of interest is a skin hyperpigmentation condition.

6. The method of claim 1, wherein the plurality of up-regulated and down-regulated genes in the biased condition signature all have a p-value of 0.05 or less.

7. The method of claim 1, further comprising determining a connectivity score for each of the instances in the database, and if the instance has a negative connectivity score, a perturbagen associated with the instance is identified as a putative skin agent having potential efficacy in the treatment of the skin condition.

8. The method of claim 1, wherein the benchmark skin care agent is selected from niacinamide, resorcinol, kojic acid, arbutin, deoxy-arbutin, vitamin C compounds, vitamin E compounds, sulfhydryl compounds, ellagic acid, glucosamine, N-acetyl glucosamine, tunicamycin, protease inhibitors, N-undecylenoyl phenylalanine, retinoids, hexamidine, fluocinolone acetonide, hydroquinone, tretinoin, hydrocortisone, phytosterol, glycyrrhetinic acid, tranexamic acid, chamomile extract, salicylic acid, alpha hydroxy acids, alpha-keto acids, and adenosine monophosphate, a mixture of fluocinolone acetonide, hydroquinone, and tretinoin, and combinations thereof.

9. The method of claim 1, wherein the biased condition signature includes 50 to 200 up-regulated genes and 50 to 200 down-regulated genes.

10. The method of claim 1, wherein constructing at least one of the condition signature and the benchmark signature comprises extracting messenger RNA (mRNA) from a plurality of skin cells and hybridizing the mRNA to a microarray.

11. The method of claim 1, wherein constructing at least one of the condition signature and the benchmark signature comprises extracting mRNA from a plurality of skin cells, reverse transcribing the mRNA to cDNA and hybridizing the cDNA to a microarray.

12. A method of making a skin care composition, comprising:
a) constructing a gene expression signature from a human skin tissue sample that exhibits a skin condition of interest;
b) constructing a benchmark signature for a type of cell present in the skin tissue sample;
c) filtering the condition signature through the benchmark signature to provide a biased condition signature, wherein the biased condition signature consists of a plurality of up-regulated genes and a plurality of down-regulated genes;
d) querying a database of instances with the biased condition signature, wherein each instance is associated with a skin care agent;
e) querying a database of instances with the biased condition signature, wherein each instance is associated with a skin care agent;
f) generating a connectivity score for each instance;
g) identifying a skin care agent associated with an instance in the database as a skin care agent for treating the skin condition of interest when the connectivity score of the instance has a negative correlation to the biased condition signature; and
h) mixing the skin care agent with dermatologically acceptable carrier to provide a skin care composition for treating the skin condition of interest.

13. The method of claim 12, wherein the skin condition of interest is a hyperpigmentation condition.

14. The method of claim 12, further comprising mixing an additional skin care agent into the skin care composition.

15. The method of claim 12, wherein the type of cells is selected from keratinocytes, fibroblasts, melanocytes, and melanoma cells.

16. A system for identifying connections between a cosmetic agent and at least one gene associated with a skin aging condition, comprising:
a) a computer readable medium having stored thereon a plurality of instances, a skin hyperpigmentation gene expression signature, and a benchmark signature, wherein each instance comprises an ordered list of identifiers representing a plurality of up-regulated and a plurality of down regulated genes differentially expressed in response to contact between a cosmetic agent and a human fibroblast cell or a human keratinocyte cell, and the skin hyperpigmentation gene expression signature and the benchmark signature each comprise one or more lists of identifiers representing a plurality of up-regulated genes and a plurality of down-regulated genes associated with a skin hyperpigmentation condition; and
b) a first computing device comprising computer-readable instructions that cause the computing device to:
i) access the hyperpigmentation gene expression signature and the benchmark signature stored on the computer readable medium;
ii) filter the hyperpigmentation gene expression signature through the benchmark signature to construct a biased skin condition signature;
iii) access the instances stored on the computer readable medium;
iv) compare each of the instances to the biased skin condition signature, wherein the comparison comprises comparing each identifier in the biased skin condition signature list(s) with the position of the same identifier in the instance list; and
v) assign a connectivity score to each of the plurality of instances based on the comparison in (iv).

17. The system of claim 16, further comprising a microarray having a plurality of probes selected to hybridize to a polynucleotide extracted from a fibroblast or a keratinocyte or derivative thereof.

18. The system of claim 17, wherein the polynucleotide is mRNA and the derivative is cDNA.

19. The system of claim 17, further comprising a microarray scanner for scanning the microarray and translating the plurality of hybridized probes to gene expression data.
